(19) United States
(12) Patent Application Publication
(10) Pub. No.: US 2005/0107868 A1
(43) Pub. Date: May 19, 2005

(54) Scaffold for tissue engineering, artificial blood vessel, cuff, and biological implant covering member

(75) Inventors: Yasuhide Nakayama, Toyonaka-shi (JP); Eisuke Tatsumi, Osaka-shi (JP); Yasushi Nemoto, Fujisawa-shi (JP)

Correspondence Address: SUGHRUE MION, PLLC, 2100 PENNSYLVANIA AVENUE, N.W., SUITE 800, WASHINGTON, DC, 20037, US

(73) Assignees: JAPAN as represented by President of NATIONAL & CARDIOVASCULAR CENTER; BRIDGESTONE CORPORATION

(21) Appl. No.: 10/950,620

(22) Filed: Sep. 28, 2004

Related U.S. Application Data
(63) Continuation of application No. PCT/JP03/03594, filed on Mar. 25, 2003.

(30) Foreign Application Priority Data
Mar. 28, 2002 (JP) 2002-091793
Sep. 5, 2002 (JP) 2002-259849
Sep. 5, 2002 (JP) 2002-259848

(51) Int. Cl. A61F 2/06; A61F 2/02

(52) U.S. Cl. 623/1.39; 606/151; 623/1.41; 623/23.76; 435/396

(57) ABSTRACT

The invention provides a porous scaffold for tissue engineering which allows easy cell engraftment and cell culture and thus enables stable organization and an artificial blood vessel which exhibits high patency rate even if the inner diameter is small. The scaffold for tissue engineering is made of thermoplastic resin which forms a porous three-dimensional network structure having communication property, wherein the porous three-dimensional network structure has an average pore diameter of from 100 to 650 μm and an apparent density of from 0.01 to 0.5 g/cm³. The artificial blood vessel is composed of this scaffold.

The invention provides a cuff which allows easy infiltration of cells from living subcutaneous tissues, easy engraftment of cells, and neovascularization of capillary vessels so as to obtain robust bonding with subcutaneous tissues and, as a result, ensures separation of a wounded portion from the outside, thereby blocking exacerbation factors such as bacterial infection on healing and inhibiting progression of downgrowth. That is, the invention provides a cuff with none or little infection trouble such as tunnel infection. The cuff comprises a porous three-dimensional network structure which is made of thermoplastic resin or thermosetting resin and has communication property, wherein the porous three-dimensional network structure has an average pore diameter of from 100 to 1000 μm and apparent density of from 0.01 to 0.5 g/cm³.

The invention provides a biological implant covering member which allows easy infiltration of cells from living subcutaneous tissues, easy engraftment of cells, and organization, thereby obtaining robust bonding with native tissues and therefore protecting a living body from adverse effect which may occur due to the insertion of a biological implantation member into the living body. The biological implant covering member comprises a porous three-dimensional network structure which is made of thermoplastic resin or thermosetting resin and has communication property, wherein the porous three-dimensional network structure has an average pore diameter of from 100 to 1000 μm and apparent density of from 0.01 to 0.5 g/cm³.

### CROSS REFERENCE TO RELATED APPLICATION

This is a continuation application of PCT/JP03/03594 filed on Mar. 25, 2003.

### TECHNICAL FIELD

The present invention relates to a scaffold material for tissue engineering, an artificial blood vessel, a cuff and a biological implant covering member.

### BACKGROUND ART

The present invention relates, in the first place, to a porous scaffold for tissue engineering which allows easy cell engraftment and cell culture and thus enables stable organization, and to an artificial blood vessel using this scaffold. The scaffold and the artificial blood vessel of the present invention are effectively used not only for basic studies on biotechnologies, but also for biomedical materials used as artificial bone structure substrates for substitute medicine by an artificial internal organ or for regenerative medicine by tissue engineering, and especially, for an artificial blood vessel which can exhibit high patency rate even if the inner diameter is small, less than 6 mm, as the endothelial cells have a nature of being engrafted all over the luminal surface.

Conventionally, as a scaffold material for tissue engineering, substrates, such as polystyrene dish (schale) or polyester mesh, coated by an extracellular matrix such as collagen are employed commonly in monolayer culture. As another culture form other than monolayer culture, there are spheroids by shake culture or embedding culture using collagen gel. Especially the embedding culture using collagen gel is advantageous because it enables in vivo culture, that is, causes a growth of cells in three dimensional structure, thus enabling basic studies on cell function while it was insufficient in monolayer culture.

In conventional artificial blood vessels, tubes made of polyester resin mesh or PTFE resin mesh have been in practical use from a long time ago, and works challenging for smaller caliber or for better patency rate has been proceeding. Primary techniques discussed until today are segmented polyurethane tubes which have been employed as antithrombotic material in practical use and artificial blood vessel material having a surface to which an antithrombotic material such as heparin is fixed using graft chain and the like.

Collagen gels for embedding culture do not have a porous structure such as three-dimensional network structure, and there remains a problem that it is impossible to obtain uniform cell engraftment on the whole surface or impossible to adjust the distribution of engraftment are not achieved. Although methods employing salt or bubbles are known as method of preparing a porous material having a three-dimensional network structure, any has difficulty in strictly and discretionary adjusting the pore diameter and pore density, so a scaffold comprising appropriate three-dimensional network structure is still not fulfilled.

Cell engraftment structure achieved by collagen gel embedding culture can not used for applications to be subjected to mechanical load such as artificial blood vessel while it is available for evaluating cell function because collagen gel as the scaffold thereof does not have physical strength.

Though artificial blood vessels as alternative materials for autologous blood vessels are used in clinical application broadly, smaller diameter artificial blood vessels have poor patency rate. Therefore for the current situation, autologous vein transplantations are still employed for coronary bypass operations, peripheral artery reconstorations requiring smaller diameter blood vessels. For the present primary techniques discussing smaller diameter pursuing only antithrombogenicity, only a pannus is formed in these conventional artificial blood vessel, but endodermis would not be formed. Accordingly, artificial blood vessels having smaller diameter have low patency rate. In addition, since wall does not have a hole through which the cell would enter, even if the pannus extends from the inosculated part, it would not be bonded to the wall and would float and many cases of resulting occlusion of blood vessels have been reported.

The invention, in the second place, relates to a cuff which enables cell penetration from the native tissue and enables a robust bonding to the native tissue, and especially relates to a cuff effective for blood circulation method by ventricular assist device, which is a treatment implanting a cannula or catheter subcutaneously, peritoneal dialysis therapy, central intravenous infusion nutrition, and for the implant part of living skin for such as transcannula DDS, transcatheter DDS, or the like.

The recently developed therapy such as ventricular assist device or peritoneal dialysis employs cannula or catheter which needs insertion under the skin and placement within the living body unlike urethra catheters, transgastrointestinal tract nutrition, and management of airway. If the placement within the living body would be of long period, for separation of living body from outside of the body and preventing intrusion of germs within the living body or evaporation of body fluid, a cuff (also said as skin cuff) would be used to artificially seal the insertion point. Conventionally, in blood circulation method by ventricular assist device, fabric velour typically made of polyester fiber would be tied around the inserting cannula, and fixing by suturing the fabric velour and subcutaneous tissue to place the cannula. Also in peritoneal dialysis, fabric velour made of polyester fiber or the like would be fixed as a cuff at the location of insertion under the skin of catheter, and subcutaneous tissue would be sutured as the cuff being oppressed to place the catheter. There is fabric velour impregnated with collagen and objected for robust bonding. In addition, there are methods fixing a cuff, which is made of a biocompatible material, to subcutaneous tissue of the insertion point.

However, in the blood circulation method using ventricular assist device, since it is a therapy assisting the blood circulation by a pulsating pump set outside the body of the patient, vibrations corresponding to 1.5 Hz from the pulsating pump are transmitted to the cannula. In other words, the insertion point of the cannula always undergoes dynamic load by vibrations. Moreover, stress occurs to denude the adhesive interface between the subcutaneous tissue and the cuff by movement of the cannula while the patient moves his or her body position or the disinfection process to the insertion point. Troubles that would be caused due to these stresses causing a lowering of adhesion between the cuff and the subcutaneous tissue include, as typical trouble, infection trouble such as tunnel infection. In cases of ventricular assist device therapy, such infection trouble experiences are being very frequent. Under existing conditions that there are a lot of cases that therapy has to be aborted due to bacterial infection not due to cardiac failure, it may be said that the therapy needs an urgent task of developing a cuff capable of preventing bacterial infection.

In peritoneal dialysis in which a catheter is inserted under the skin and placed for a long period, there remains a momentous problem on cuffs. That is, in this therapy, the catheter is placed within the abdominal cavity in order to inject or discharge dialyzing fluid. However, the living body recognizes the catheter as a foreign substance and therefore acts to reject the catheter so that the adhesion between the subcutaneous tissue and the catheter would not be made, thus causing a downgrowth phenomenon that the skin surface barges into the abdominal cavity along the catheter. This pocket of downgrowth makes reach of disinfectant difficult, triggering inflammation of skin or tunnel infection and finally resulting in induction of peritonitis. Considering reports that patients experiencing frequent peritonitis of Pseudomonas aeruginosa increased incidence of SEP (sclerosing encapsulating peritonitis), the improvement of cuff to prevent infection would be a momentous object on the peritoneal dialysis therapy.

As described above, cuffs consisting primarily of collagen have been developed. However, in the case of this kind of cuff, the volume would decrease by absorbing liquid such as normal saline solution, alcohol, Isodine, blood and/or body fluid so that it is difficult to breed the subcutaneous tissue on the location of the insertion of the catheter. As a result, inhibiting effect of downgrowth is not attained.

The invention, in the third place, relates to a biological implant covering member, which covers the surface of a biological implantation member such as artificial heart valve, artificial heart valve ring, artificial blood vessel, artificial breast, artificial bone, artificial joint and artificial heart or other associated parts thereof, thereby reducing the foreign-body reaction in the living body.

Conventionally, constituent materials for biological implantation member such as artificial heart valve, artificial heart ring, artificial blood vessel, artificial breast, artificial bone, artificial joint and artificial heart, and the like and other associated parts thereof have been studied mainly with a focus on materials that generate no or little eluate and is chemically inactive causing no or little stimulation to the surrounding tissue, and would be immunologically neglected by the living body. Examples of those materials include metal materials such as titanium, stainless steel and platinum, ceramic materials such as hydroxyapatite and polymeric materials such as polytetrafluoroethylene, polyester and polypropylene, and are in practical use for various applications. For instance, metallic materials are used for intravascular stent, bone fixing bolt, and artificial joint. Ceramic materials are used as, for example, artificial joints and artificial bones for filling or substituting deficient parts of joints and bones. Polymeric materials have been put in practical use as artificial blood vessel for retaining blood flow after aneurysmectomy, suture thread for suturing a part which needs incision once again for enabling suture removal, artificial trachea, and artificial breast for prosthetic surgery of the lost breast caused by breast canser incision or for breast enlargement in plastic surgery.

Metallic materials for biological implantation, for example, a stent to be placed within the blood vessel would consist primarily of good rust prevention stainless steel. However, in case of long period placement within the blood vessel, the stent is constantly exposed to various electrolytes, protein, lipid containing blood so that rust would form and possibly result in irritation of the surrounding tissue.

Mainstream artificial breasts in practical use are made of a silicone bag filled with normal saline solution and the like. However, the loculated collagen tissue would be thickened and contract on the surface after subcutaneous implant, and in this case, there was a problem that the silicone bag would deform within the living body, compressing the surrounding tissue, evoking inflammation reaction, or making breast cancer to recur.

As for an artificial trachea, products composing of silicone tube have been put in practical use, however, it has no affinity for living tracheas, and had a problem that it would detach during long-term implant or cause infection on the boundary face.

In the case of implantable artificial heart, for example, the vibrational inertia of the driving motor results in a problem of pocket infection, which is occurred by the inflammation or infection on the native tissue boundary surface.

### SUMMARY OF THE INVENTION

It is an object of the invention according to the first aspect to provide a scaffold material for tissue engineering which comprises a homogeneous porous body having a three-dimensional network structure, allows cells to be uniformly engrafted all over the inside of the porous body thereof, is excellent in physical strength, and is effectively used not only for basic studies on biotechnologies, but also for an artificial blood vessel which can exhibit high patency rate for a long period of time even if the inner diameter is small, less than 6 mm, and to provide an artificial blood vessel using this scaffold for tissue engineering.

A scaffold material for tissue engineering of the present invention is a scaffold material for tissue engineering made of thermoplastic resin forming a porous three-dimensional network structure having communication property, wherein the porous three-dimensional network structure has an average pore diameter of from 100 to 650 μm and an apparent density of from 0.01 to 0.5 g/cm³.

Since the scaffold for tissue engineering of the present invention has the porous three-dimensional network structure made of thermoplastic resin and having the certain average pore diameter and the certain apparent density mentioned above, cells and collagen suspension are allowed to easily penetrate into pores of the porous three-dimensional network structure. Therefore, cells can be seeded all over the porous three-dimensional network structure. For example, an artificial peritoneal composed of two layers of mesothelial cell and fibrocyte can be obtained. It is expected that the scaffold is used for analyzing the mechanism of glycosylation in peritoneal dialysis and for basic study of the dialysis. When the scaffold for tissue engineering is used as an artificial blood vessel, vascular endothelial cell can be present in the luminal surface of the artificial blood vessel so that occlusion hardly occurs. As a result, it is possible to achieve an artificial blood vessel of small diameter.

The artificial blood vessel of the present invention is composed of the scaffold of the present invention, can exhibit high patency rate even if the inner diameter is small, less than 6 mm, and is therefore effectively applied to coronary bypass operations, peripheral arterial reconstoration, and the like.

It is an object of the invention according to the second aspect to provide a cuff which allows easy infiltration of cells from living subcutaneous tissues, easy engraftment of cells, and neovascularization of capillary vessels so as to obtain robust bonding with subcutaneous tissues, thereby inhibiting progression of downgrowth, and therefore has none or little risk of infection trouble such as tunnel infection.

A cuff of the present invention comprises a porous three-dimensional network structure which is made of thermoplastic resin or thermosetting resin and has communication property, wherein the porous three-dimensional network structure has an average pore diameter of from 100 to 1000 μm and apparent density of from 0.01 to 0.5 g/cm³.

Since the cuff of the present invention has a porous three-dimensional network structure which is made of thermoplastic resin or thermosetting resin and has communication property and which has the certain average pore diameter and the certain apparent density mentioned above, the cuff allows easy infiltration of cells into pores of the porous three-dimensional structure and easy engraftment of cells so as to obtain robust bonding with living tissues.

It is an object of the invention according to the third aspect to provide a biological implant covering member which allows easy infiltration of cells from living subcutaneous tissues, easy engraftment of cells, and organization, thereby obtaining robust bonding with native tissues and therefore protecting a living body from adverse effect which may occur due to the insertion of a biological implantation member into the living body.

A biological implant covering member of the present invention comprises a porous three-dimensional network structure which is made of thermoplastic resin or thermosetting resin and has communication property, wherein the porous three-dimensional network structure has an average pore diameter of from 100 to 1000 μm and apparent density of from 0.01 to 0.5 g/cm³.

Since the biological implant covering member of the present invention has a porous three-dimensional network structure which is made of thermoplastic resin or thermosetting resin and has communication property and which has the certain average pore diameter and the certain apparent density mentioned above, the biological implant covering member allows easy infiltration of cells into pores of the porous three-dimensional structure, easy engraftment of cells, and neovascularization of capillary vessels so as to obtain robust bonding with native tissues.

The biological implant covering member of the present invention has a porous three-dimensional network structure which enables penetration and engraftment of cells and neovascularization of capillary vessels.

Therefore, a biological implant covering member of the present invention is used to cover the surface of a biological implantation member such as artificial heart valve, artificial heart valve ring, artificial blood vessel, artificial breast, artificial bone, artificial joint and artificial heart or other associated parts thereof, thereby reducing the foreign-body reaction against the biological implantation member by peripheral tissues.

The biological implantation member means an object to be implanted into a living body and includes a system composed of various parts. Examples are, as for an artificial heart system, an actuator (energy converter) as an in vivo driving unit, left and right blood pumps as pumps, an atrial cuff, an atrial connector, an artery graft and an artery connector, an in vivo secondary coil in a percutaneous energy transfer system, an in vivo unit in a percutaneous information transfer system, an in vivo battery in a buttery system, an in vivo control unit in a control system, and a compliance chamber, a volume displacement chamber, and a bent tube in a volume displacement system. Beside these, there are examples a device composed of a large number of parts such as in vivo unit connecting cable and connector. In the present invention, all of these are called as biological implantation member.

The biological implant covering member may be used for purposes other than clinical purposes and may be used to cover the outer surface of a transmitter to be implanted into an animal body for the purpose of ecological survey, thereby reducing the foreign-body reaction.

### BRIEF DESCRIPTION OF THE DRAWINGS

FIG. 1 is a SEM (scanning electron microscope) picture (×20) showing the entire tubular structure of a scaffold material made in Example 1;

FIG. 2 is a stereoscopic microscope picture (×100) showing a fine structure inside the tubular structure of the scaffold made in Example 1;

FIG. 3 is a SEM picture (×20) showing a surface layer of the inner wall of the tubular structure of the scaffold made in Example 1;

FIG. 4 is a SEM picture (×20) showing a surface layer of the outer periphery of the tubular structure of the scaffold made in Example 1;

FIG. 5 is a SEM picture (×10) showing a porous three-dimensional network structure containing cells made in Example 2 after three days of incubation;

FIG. 6 is an optical microscope picture (×10) showing that interior tissues are engrafted on entire surface even after one week of additional incubation in Example 2,

FIG. 7 is a picture showing a scene where bloodstream is obtained by artificial blood vessels, thus occurring heartbeat in Example 3;

FIG. 8 is a picture showing that no blood clot is generated in the inside of the artificial blood vessels after one week from implantation in Example 3;

FIG. 9 is a SEM picture (×50) showing a surface layer of a tubular structure made in Comparative Example 1;

FIG. 10 is a SEM picture (×50) showing a fine structure inside the tubular structure made in Comparative Example 1;

FIG. 11 is an optical microscope picture (×10) showing a tubular structural material containing cells made in Comparative Example 2 after three days of incubation;

FIG. 12 is a SEM picture (×50) showing a surface of a tissue contact side of a cuff made in Example 4;

FIG. 13 is a SEM picture (×50) showing an inner section of the cuff made in Example 4;

FIG. 14 is a distribution chart obtained by measuring distribution in pore diameter of the cuff made in Example 4;

FIG. 15 is a picture just after an operation of implanting a cuff made in Example 4 into an incised part of chest of a goat and fixing the cuff by suturing subcutaneous tissues; and

FIG. 16a is an enlarged picture showing tissues surrounding the test piece after the cuff made in Example 4 was implanted into the incised part of chest of a goat for two weeks and then removed and FIG. 16b is an enlarged picture showing tissues surrounding the test piece in case that the same test was conducted using a fabric for comparison.

### DESCRIPTION OF THE PREFERRED EMBODIMENTS

Hereinafter, embodiments of scaffolds for tissue engineering and artificial blood vessels of the present invention will be studied in detail.

The scaffold for tissue engineering of the present invention is made of thermoplastic resin forming a three-dimensional network structure. The three-dimensional network structural layer is a porous three-dimensional network structural layer which has an average pore diameter of from 100 to 650 μm and an apparent density of from 0.01 to 0.5 g/cm³ and has communication property, that is, has continuous pores. The three-dimensional network structural layer may be formed to have allover similar configuration from the inner wall to the outer wall and may be formed such that the configuration at portions near the inner wall is different from the configuration at portions near the outer wall. In addition, the average pore diameter and the apparent density may vary partially. For example, the average pore diameter may vary gradually from the inner wall to the outer wall, that is, the three-dimensional network structural layer may have anisotropy.

It should be noted that the “porous three-dimensional network structural layer” is referred as—porous three-dimensional network structure—hereinafter.

As for the three-dimensional network structure composed of the thermoplastic resin, the average pore diameter is from 100 to 650 μm and the apparent density is from 0.01 to 0.5 g/cm³ as mentioned above. The average pore diameter is preferably from 100 to 400 μm, more preferably from 100 to 300 μm. The apparent density of from 0.01 to 0.5 g/cm³ can provide well cell engraftment, excellent physical strength, and elastic characteristics similar to that of living body. The apparent density is preferably from 0.01 to 0.2 g/cm³, more preferably from 0.01 to 0.1 g/cm³.

As for the concept of the average pore diameter, the distribution of pore diameters is preferably monodisperse and higher contribution ratio of pores of 150-300 μm diameter (this pore size is important in allowing cell infiltrate) is better. The contribution ratio of pores of 150-300 μm diameter is 10% or more, preferably 20% or more, more preferably 30% or more, particularly preferably 40% or more, especially preferably 50% or more. Since such contribution ratio of pores of 150-300 μm diameter enables cells to easily invade and allows the invaded cell to easily adhere and grow, the three-dimensional network structure having such contribution ratio is effective for application as a scaffold material and an artificial blood vessel.

The contribution ratio of pores of 150-300 μm diameter in the average pore diameter of the porous three-dimensional network structure denotes a ratio of the number of pores of 150-300 μm diameter relative to the number of all pores in a measuring method of average pore diameter in Example 1 described later.

By using this porous three-dimensional network structure having the aforementioned average pore diameter, apparent density, and pore diameter distribution, an excellent scaffold can be obtained which allows cell/collagen suspension culture solution to easily penetrate into pores and allows easy adhesion and growth of cells to porous layers. In case that the scaffold is formed in a tubular shape, cells can be engrafted all over from the inner wall to the outer periphery, thereby achieving an artificial blood vessel at a low risk of occlusion and with high patency rate.

Examples of the thermoplastic resin composing the scaffold for tissue engineering of the present invention include polyurethane resin, polyamide resin, polylactide resin, polyolefin resin, polyester resin, fluorocarbon resin, acrylic resin, methacrylic resin, and derivatives thereof. These may be used alone or in admixture of two or more. Among these, polyurethane resin is preferable and segmented polyurethane resin capable of providing an artificial blood vessel which is excellent in antithrombogenicity and physical property is especially preferable.

The segmented polyurethane resin is prepared synthetically from three components: a polyol, a diisocyanate, and a chain elongation agent and thus has elastomeric characteristics according to a so-called block polymer structure having hard segments and soft segments within molecule. Therefore, the scaffold and the artificial blood vessel made using this segmented polyurethane resin can be formed into a tubular structure which exhibits an S-S curve (characteristics of high compliance and low elasticity at low blood pressure range and low compliance and high elasticity at high blood pressure range) approximate to a living blood vessel in elastic dynamics and is excellent in antithrombogenicity and physical property.

By using a thermoplastic resin having hydrolyzable property or biodegradability, a resin substrate is gradually dissolved and absorbed after implantation of an artificial blood vessel into a living body and can be finally removed from the living body with leaving engrafted cells.

In the porous three-dimensional network structure made of the thermoplastic resin, one or more selected from a group composing of collagen Type I, collagen Type II, collagen Type III, collagen Type IV, atelocollagen, fibronectin, gelatin, hyaluronic acid, heparin, keratin acid, chondroitin, chondroitin sulfate, condroitin sulfate B, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone may be held. Further in the porous three-dimensional network structure, cytokines of one or more kinds selected from a group composing of fibrocyte growth factor, interleukin-1, tumor growth factor-β, epidermal growth factor, and diploidic fibrocyte growth factor may be held. Furthermore in the porous three-dimensional network structure, cells of one or more kinds selected from a group composing of embryo-stem cell, vascular endothelial cell, mesodermal cell, smooth muscle cell, peripheral vessel cell, and mesothelial cell may be attached. The embryo-stem cell may be dividing cell.

The scaffold for tissue engineering of the present invention enables its skeleton made of the thermoplastic resin constructing the porous three-dimensional network structure to be provided with fine pores. These fine pores make the skeleton to have complex irregular surface not smooth surface. The irregular surface is effective in holding collagen and cell growth factor, resulting in increased cell engraftment. These fine pores are outside the concept of calculating the average pore diameter of the porous three-dimensional network structure as employed in the present invention.

The configuration of the scaffold for tissue engineering of the present invention is not particularly limited. If taking a form of tubular structure, the scaffold can be used as an artificial blood vessel.

In this case, the tubular structure is 0.3-15 mm in inner diameter and 0.4-20 mm in outer diameter, preferably 0.3-10 mm in inner diameter and 0.4-15 mm in outer diameter, further preferably 0.3-6 mm in inner diameter and 0.4-10 mm in outer diameter, particularly preferably 0.3-2.5 mm in inner diameter and 0.4-10 mm in outer diameter, especially preferably 0.3-1.5 mm in inner diameter and 0.4-10 mm in outer diameter. Even in a case of such small diameter artificial blood vessel, high patency rate can be maintained.

The artificial blood vessel of the present invention composed of the scaffold of the present invention may be a tubular structure of which outside is covered by another tubular structure. In case that the impregnation density of collagen and the like into the scaffold of the present invention is low and/or that the thickness of the scaffold is small, a covering layer by this tubular structure prevents leakage of blood for a certain period after implantation and is absorbed in the living body and is thus removed when there is no more possibility of blood leakage after sufficient adhesion and engraftment of cells. The tubular structure for covering is not particularly limited and, for example, may be a tube made of one or more selected from a group composing of chitosan, polylactide resin, polyester resin, polyamide resin, polyurethane resin, fibronectin, gelatin, hyaluronic acid, keratin acid, chondroitin, chondroitin sulfate, condroitin sulfate B, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone, cross-linked collagen, and fibroin. The thickness (difference between the outer diameter and the inner diameter) of the tubular structure for covering such as a chitosan tube is preferably in the range of 5-500 μm.

Though the artificial blood vessel of the present invention has novelty in that high patency can be achieved so as to ensure stable blood flow even in a case of a small-diameter vessel that has never been achieved by any conventional technique, the artificial blood vessel of the present invention can be adapted to a large-diameter vessel having an inner diameter of 6 mm or more without any problems.

Hereinafter, an example of the method of producing a porous three-dimensional network structure made of a thermoplastic polyurethane resin for forming a scaffold material or a tubular structure as an artificial blood vessel of the present invention will be described. However, the method of producing a porous three-dimensional network structure made of a thermoplastic polyurethane resin according to the present invention is not limited to the following described method at all. According to the following method, thermoplastic resin substrates of three-dimensional network structure of various configurations, such as a plane substrate, required as the scaffold for tissue engineering can be prepared.

To prepare a porous three-dimensional network structure made of a thermoplastic polyurethane resin, first a polymer dope is prepared by mixing a polyurethane resin, a water-soluble polymer compound, as will be described later, as a pore forming agent, and an organic solvent as a good solvent for the polyurethane resin. Specifically, after the polyurethane resin is mixed into the organic solvent to have a homogeneous solution, a water-soluble polymer compound is mixed and dissolved into this homogeneous solution. Examples of the organic solvent include N,N-dimethylformamide, N-methyl-2-pyrrolidinone, and tetrahydrofuran. However, the organic solvent to be used is not limited thereto and may be any organic solvent capable of solving the thermoplastic polyurethane resin. In addition, the polyurethane resin may be dissolved by heat with reduced amount of organic solvent or without organic solvent and the pore forming agent may be mixed to the dissolved polyurethane resin.

Examples of the water-soluble polymer compound as pore forming agent include polyethylene glycol, polypropylene glycol, polyvinyl alcohol, polyvinyl pyrrolidone, alginic acid, carboxymethyl cellulose, hydroxypropyl cellulose, methyl cellulose, and ethyl cellulose. However, the water-soluble polymer compound to be used is not limited thereto and may be any water-soluble polymer compound capable of being homogeneously dispersed with the thermoplastic resin to form a polymer dope. In addition, depending on the kind of the thermoplastic resin, the polymer compound is not limited to water-soluble ones. For example, lipophilic compounds such as phthalate ester and paraffin, and inorganic salts such as lithium chloride and calcium carbonate may be used. It is also available to use crystal-nucleation agent for polymer so as to generate secondary particles during coagulation, that is, to encourage skeletal formation of porous body.

The polymer dope prepared from the thermoplastic polyurethane resin, the organic solvent, and water-soluble polymer compound is dipped in coagulation bath containing a poor solvent of thermoplastic polyurethane resin so as to extract and remove organic solvent and water-soluble polymer compound in the coagulation bath. By eliminating a part or all of organic solvent and water-soluble polymer compound, a porous three-dimensional network structural material of polyurethane resin is obtained. Examples of the poor solvent used herein include water, lower alcohol, and low carbon number ketones. The coagulated polyurethane resin is finally washed with water or the like to remove remaining organic solvent and pore forming agent.

Hereinafter, examples and comparative examples will be described, but the present invention is not limited by the following examples at all without departing from the scope of the invention.

### EXAMPLE 1

A thermoplastic polyurethane resin (MIRACTRAN E980PNAT available from Nippon Miractran Co., Ltd.) was dissolved into N-methyl-2-pyrrolidinone (reagent for peptide synthesis, NMP available from Kanto Kagaku) by using a dissolver (about 2000 rpm) at room temperature to obtain 5.0% solution (weight/weight). 1.0 kg of this NMP solution was measured and entered into a planetary mixer (PLM-2 type, capacity 2.0 liters, available from Inoue Mfg., Inc.) and was mixed with methylcellulose (reagent, 25 cp grade, available from Kanto Kagaku) of an amount corresponding to the amount of polyurethane resin at a temperature of 40° C. for 20 minutes. With the agitation being continued, the defoaming was conducted by reducing the pressure to 20 mmHg (2.7 kPa) for 10 minutes, thereby obtaining polymer dope.

A tube forming jig was prepared which comprised cylindrical paper tube of 3.5 mmφ in inner diameter, 4.6 mmφ in outer diameter, and 60 mm in length made of a chemical experimental paper filter (Qualitative filter paper No. 2, available from Toyo Roshi Kaisha, Ltd.), a mandrel of 1.2 mmφ in diameter made of SUS440, and a cylindrical airtight stopper made of biomedical polypropylene resin capable of fixing the mandrel at the center of the paper tube. The polymer dope was injected into the tube forming jig by using a needle of 23 gauges. After that, the tube forming jig was tightly stopped and then entered into methanol under refluxing condition. The refluxing was continued for 72 hours to extract and remove NMP solution from inside through the surface of the paper tube, whereby the polyurethane resin was coagulated. During this, the methanol was replaced with new one as needed with keeping the refluxing condition. After 72 hours, the tube forming jig was moved to a methanol bath at room temperature from the methanol under the refluxing condition without being dried. The content was extracted from the tube forming jig within the bath and was washed in purified water of the Japanese Pharmacopoeia for 72 hours to extract and remove methylcellulose, methanol, and remaining NMP. The water for washing was replaced with new one as needed. The washed content was depressurized (20 mmHg (2.7 kPa)) at room temperature for 24 hours and dried, thereby obtaining a tubular scaffold of porous three-dimensional network structure which can be used as an artificial blood vessel.

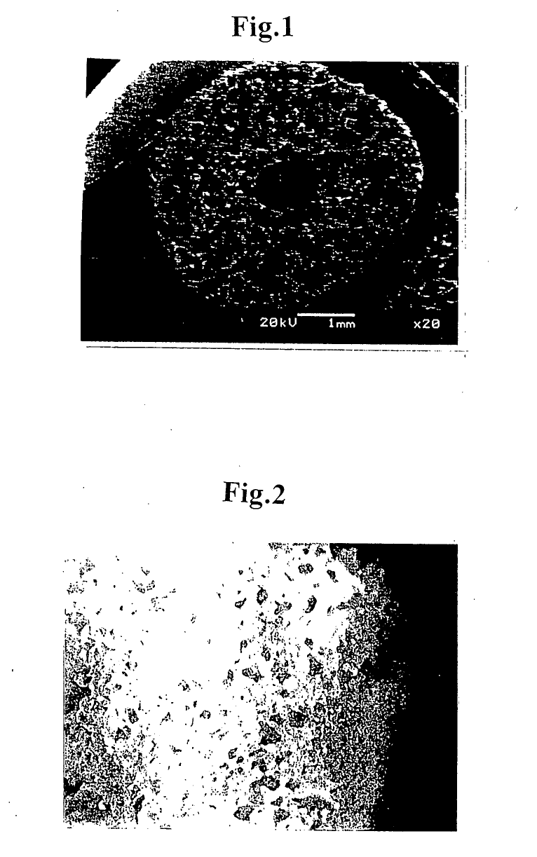

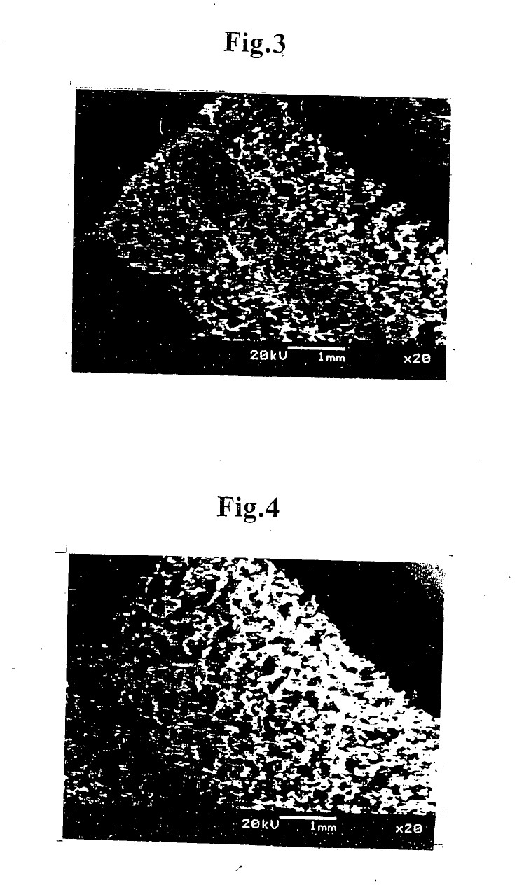

FIGS. 1 through 4 are pictures of this scaffold taken by a scanning electron microscope (SEM, JMS-5800LV, available from JEOL Ltd.) or by a stereoscopic microscope (VH-6300 available from Keyence Corporation). Apparent from FIGS. 1 through 4, the substrate of the obtained scaffold is a porous three-dimensional network structure of about 200 μm in pore diameter, 1.2 mmφ in inner diameter, and 3.2 mmφ in outer diameter in which the inside of the structure (FIG. 2), the surface layer of the inner wall (FIG. 3), and the surface layer of the outer periphery (FIG. 4) are substantially the same and is an entirely homogeneous porous body.

For the obtained scaffold, the average pore diameter and the apparent density were measured according to the following methods. In the measurements of the average pore diameter and the apparent density, specimens were cut by using a twin bladed razor (HighStainless available from FEATHER Safety Razor Co., Ltd) at room temperature.

[Measurement of Average Pore Diameter]

By using a picture of a plane (cutting surface) of specimen, cut by the twin bladed razor, taken by a stereoscopic microscope (VH-6300 available from Keyence Corporation), image processing was conducted to take respective pores on the same plane as figures surrounded by skeleton of three-dimensional network structure (using LUXEX AP available from NIRECO Corporation as an image processing unit and LE N50 available from SONY Corporation as a CCD camera for taking images) and the areas of the respective figures were measured. The areas were converted to areas of real circles. The diameters of the corresponding circles were obtained as the pore diameters. Measurement was conducted only for through pores on the same plane in disregard for micropores bored in the porous skeleton, with the result that the average pore diameter was obtained as 169±55 μm. The contribution ratio of pores of 150-300 μm diameter in the pore distribution was obtained as 71.2% so that it was recognized that the specimen was a porous body mainly having pores effective in cell adhesion.

[Measurement of Apparent Density]

The scaffold was cut into a specimen of about 10 mm in length by the twin bladed razor. The volume of the specimen was obtained from dimensions measured by a projector (V-12, Nikon). As a result that the weight was divided by the volume, the apparent density was obtained as 0.077±0.002 g/cm³.

The three-dimensional network structure as a characteristic of the present invention is a structure which is excellent in pore-to-pore communication. The water permeability as indicator of this communication property was evaluated as follows.

[Evaluation of Water Permeability]

First, a specimen of 10 mm in length was prepared by cutting the material as mentioned above. While the end of one side of the specimen was tightly stopped, a needle of 0.3 mmφ in inner diameter, 1.2 mmφ in outer diameter, 40 mm in length was inserted into the specimen at the other side in such a manner as to obtain 0.50 mm in length as the effective permeability area of a tubular body of the specimen. A silicone tube of 50 mm in length and 5 mmφ in diameter and a gator of 20 mmφ and 90 mm in length filled with 25 g of water were connected to the needle so as to measure the permeability of distilled water at a temperature of 25° C. The water permeation rate was 13.47±0.33 g/60 sec. and 24.64±0.35 g/120 sec. Since the water permeation rate with no specimen, i.e. in the unloaded state, was 13.70/60 sec. and 24.87/120 sec., it was recognized that the scaffold was a three-dimensional network structure having excellent water permeability with high communication property.

### EXAMPLE 2

DMEM (culture component) solution (containing FCS (cow embryo blood serum) 10%) of smooth muscle cells from cow's blood vessel (cell density: 6×10⁶ cells/mL) and collagen type I solution (0.3% acid solution available from Koken Co., Ltd.) are mixed in equivalent quantities while being cooled on ice, thereby preparing suspension solution of smooth muscle cells (cell density: 3×10⁶ cells/mL).

The scaffold of tubular porous three-dimensional network structure (inner diameter: 1.2 mmφ, outer diameter: 3.2 mmφ, length: 2 cm) prepared in Example 1 was clamped at its one end and the suspension solution of smooth muscle cells (1 mL) was injected at the other end into the scaffold until leaking out through a side wall of the tubular structure. All of the injection operation was conducted on ice. By repeating the injection operation several times, the collagen solution containing smooth muscle cells well penetrated all over the tubular structure including the inside thereof. After that, the clamping was cancelled, a mandrel of 1.2 mmφ made of SUS440 was inserted into the tubular body of the scaffold at the center thereof, and incubation was conducted in an incubator at a temperature of 37° C., thereby obtaining a porous three-dimensional network structure containing cells.

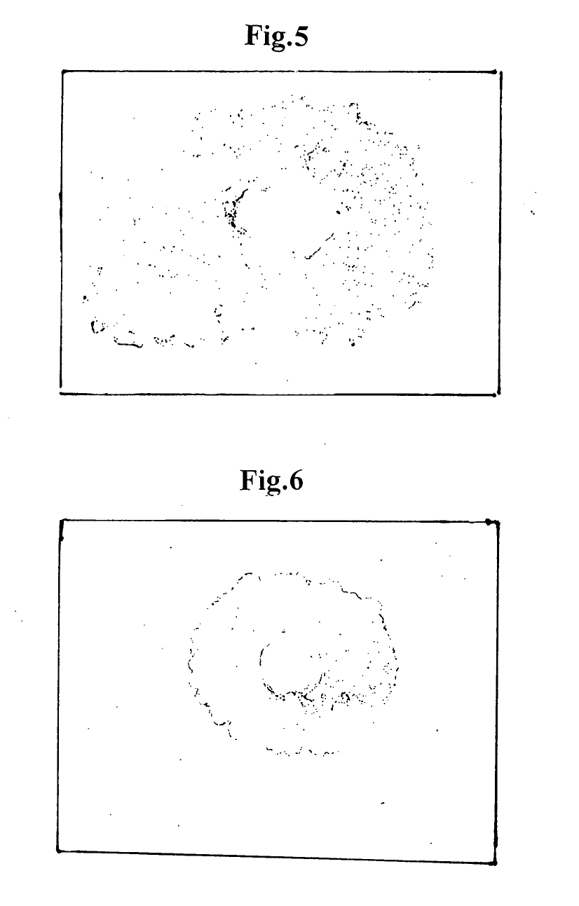

The porous three-dimensional network structure containing cell obtained as mentioned above underwent three days of incubation. FIG. 5 is a picture showing sectional tissue of the porous three-dimensional network structure observed by an optical microscope after three days of incubation. From FIG. 5, it is found that the cells are distributed all over the inside of the obtained structure. It was observed that tissues inside the structure containing cells engrafted without necrosing even after one week of additional incubation (FIG. 6).

### EXAMPLE 3

The scaffold of tubular porous three-dimensional network structure (inner diameter: 1.2 mmφ, outer diameter: 3.2 mmφ, length: 2 cm) prepared in Example 1 was clamped at its one end and the collagen type I solution (0.15 wt. %) was injected at the other end into the scaffold until the collagen solution penetrated all over the scaffold including the inside thereof. After that, the clamping was cancelled, a mandrel of 1.2 mmφ made of SUS440 was inserted into the tubular body of the scaffold at the center thereof, and the tubular structure of the scaffold was held inside an incubator at a temperature of 37° C. to make the collagen solution to gel, thereby obtaining a tubular body of which network structure was filled with collagen gel.

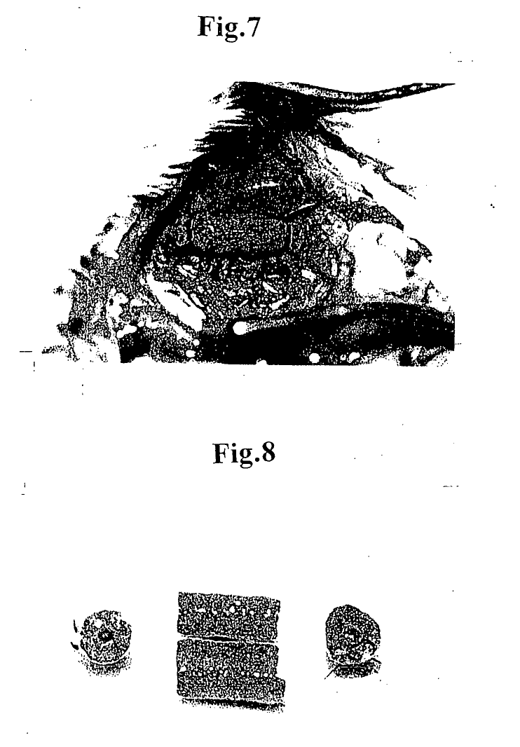

A piece of about 3 cm was exfoliated from aorta abdominalis of a rat and was clamped at its both ends to block the blood stream. After that, a middle portion of the aorta was cut. The tubular body was inserted between the cut ends of the aorta and the ends of the tubular body are connected to the corresponding cut ends. As the blood stream was reactivated after canceling the clamping of both ends, beat starts. Therefore, the tubular body functioned as an artificial blood vessel (FIG. 7). The artificial blood vessel was removed after one week. As the lumen surface of the tubular tissue body was observed, blood clot was not attached nor formed on the lumen surface so that the lumen surface was really smooth (FIG. 8).

### COMPARATIVE EXAMPLE

thermoplastic polyurethane resin (MIRACTRAN E980PNAT available from Nippon Miractran Co., Ltd.) was heated at a temperature of 60° C. to lyse into tetrahydrofuran (THF available from Wako Pure Chemical Industries, Ltd.), thereby obtaining 5.0% solution (weight/weight) thereof. 12 g of NaCl particles (having particle diameters ranging from 100 μm to 200 μm which were selected by filtering procedure) were dispersed into 16 mL of the THF solution, thus preparing suspension. A mandrel of 1.2 mmφ in diameter made of SUS440 was immersed in the suspension and was dried, whereby the periphery of the mandrel was coated with tubular coating of polyurethane containing NaCl particles. After the mandrel with coating was sufficiently dried, the mandrel was washed enough with ion-exchange water to remove NaCl contained in the tubular coating. The washed mandrel was depressurized (20 mmHg (2.7 kPa)) at room temperature for 24 hours and dried, thereby obtaining a porous tubular body of 1.2 mmφ in inner diameter and 3.2 mmφ in outer diameter.

The average pore diameter and the apparent density of this porous tubular body were measured in the same manner as Example 1. While the average pore diameter was 121±65 μm, the contribution ratio of pores of 150-300 μm diameter was 31.8%. The apparent density was 0.086±0.004 g/cm³.

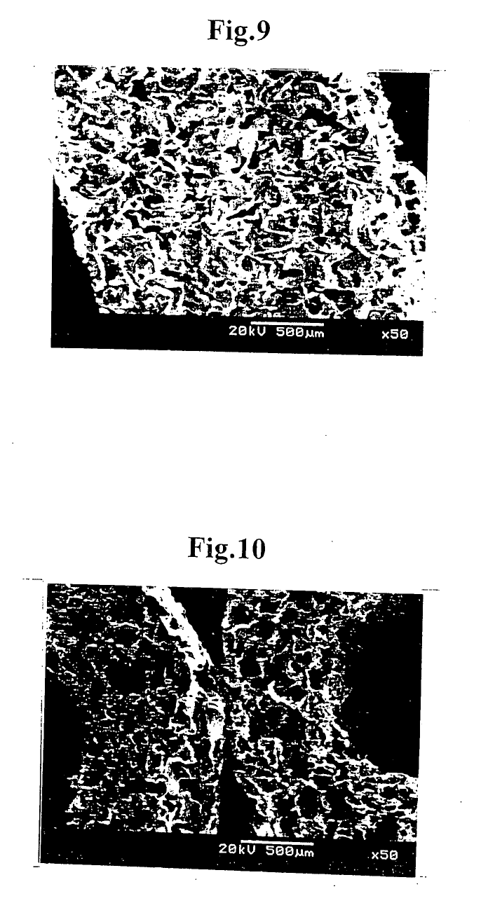

As a result of appearance observation by the SEM, while Example 1 had a three-dimensional structure in which the outer layer and the inside are the same, this comparative example had a structure in which the outer layer and the inside are quite different from each other because closely-spaced layers were generated in the outer layer (FIG. 9) and spherical pores were gathered in the inside structure so that at contact portions between adjacent pores, pore walls were provided with penetrated holes, that is, this structure was not a three-dimensional network structure (FIG. 10).

The water permeation rate was also measured in the same manner as Example 1, with the result of 11.22±0.46 g/60 sec. and 20.08±0.96 g/120 sec. These values were lower than those of Example 1. It can be concluded that this is because the communication property between pores in the outer layer is low and the closely-spaced layer in the outer layer affects.

### COMPARATIVE EXAMPLE 2

Suspension solution of smooth muscle cells (cell density: 3×10⁶ cells/mL) prepared in the same manner as Example 2 was injected into the porous tubular body (inner diameter: 1.2 mmφ, outer diameter: 3.2 mmφ, length: 2 cm) prepared in Comparative Example 1 in the same manner as Example 2. After that, incubation was conducted, thereby obtaining a tubular structural material containing cells.

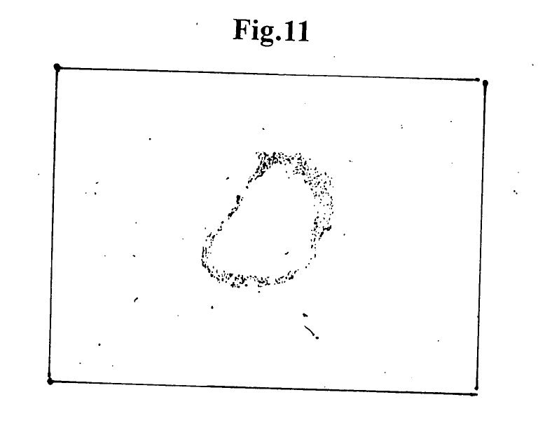

The tubular structural material containing cells obtained as mentioned above underwent three days of incubation. FIG. 11 is a picture showing sectional tissue of the tubular structural material containing cells observed by an optical microscope after three days of incubation. From FIG. 11, it is found that little cells exist inside the obtained structure and cells exist only on the luminal surface.

As described in the above, the present invention can provide a scaffold material for tissue engineering which comprises a homogeneous porous body having a three-dimensional network structure, allows cells to be uniformly engrafted all over the inside of the porous body thereof, is excellent in physical strength, and is effectively used not only for basic studies on biotechnologies, but also for an artificial blood vessel which can exhibit high patency rate for a long period of time even if the inner diameter is small, less than 6 mm, and the present invention can provide an artificial blood vessel using this scaffold for tissue engineering.

Hereinafter, preferred embodiments of the cuff of the present invention will be described in detail.

The cuff of the present invention is composed of a three-dimensional network structure having well communication property made of thermoplastic resin or thermosetting resin. The three-dimensional network structure is a porous three-dimensional network structure having an average pore diameter from 100 to 1000 μm and apparent density from 0.01 to 0.5 g/cm³. In the cutting surfaces in the depth direction, the surfaces may be entirely similar or one side surface is different from the other side surface. The average pore diameter and/or the apparent density may partially vary. For example, the average pore diameter may vary gradually from the one side surface to the other side surface, that is, the three-dimensional network structure may have anisotropy. The three-dimensional network structure may be provided, in the contact surface with native tissues, with pores having a large pore diameter which is extremely larger than the average pore diameter. It is preferable that these pores are pores having a pore diameter in the range of 500-2000 μm. These pores existing near the outer layer on the side of native tissues facilitate extracellular matrix such as collagen to homogeneously penetrate deep parts and effectively act on infiltration of cells from tissues and neocascularization of capillary vessels. It should be noted that such large diameter pores are outside the concept of calculating the average pore diameter of the porous three-dimensional network structure as employed in the present invention.

As for the porous three-dimensional network structure, the average pore diameter is from 100 to 1000 μm and the apparent density is from 0.01 to 0.5 g/cm³. The average pore diameter is preferably from 200 to 600 μm, more preferably from 200 to 500 μm. The apparent density in the range of from 0.01 to 0.5 g/cm³ can provide well cell engraftment, excellent physical strength, and elastic characteristics similar to subcutaneous tissues when cells infiltrate, are sufficiently grown and tightly interconnected. The apparent density is preferably from 0.05 to 0.3 g/cm³, more preferably from 0.05 to 0.2 g/cm³.

As for the distribution of pore diameter with the same average pore diameter, higher contribution ratio of pores of 150-400 μm diameter that is important in allowing cell infiltrate is better. The contribution ratio of pores of 150-400 μm diameter is 10% or more, preferably 20% or more, more preferably 30% or more, particularly preferably 40% or more, especially preferably 50% or more. Such contribution ratio is preferable because it enables cells to easily invade and allows the invaded cell to easily adhere and grow.

The contribution ratio of pores of 150-400 μm diameter in the average pore diameter of the porous three-dimensional network structure denotes a ratio of the number of pores of 150-400 μm diameter relative to the number of all pores in a measuring method of average pore diameter in Example 4 described later.

The porous three-dimensional network structure having the average pore diameter, the apparent density, and the distribution of pore diameters as mentioned above allows easy infiltration of cells into pores and allows easy adhesion and growth of cells onto the porous three-dimensional network structure, thereby constructing capillary vessels. Therefore, with this porous three-dimensional network structure, an excellent cuff which can provide a robust bonding between subcutaneous tissue and a catheter or cannula at a portion of the cuff insertion can be obtained.

The porous three-dimensional structure may have a thickness ranging from 0.2 mm to 500 mm. The thickness is preferably from 0.2 to 100 mm, more preferably from 0.2 to 50 mm, particularly preferably from 0.2 to 10 mm, especially preferably from 0.2 to 5 mm. Such a thickness as mentioned provides a high level of satisfaction in physical strength required as a cuff, infiltrate of cells, organization, bonding with subcutaneous tissue, and antibacterial property.

The thermoplastic resin or thermosetting resin composing the porous three-dimensional network structure may be one or more of polyurethane resin, polyamide resin, polylactide resin, polyolefin resin, polyester resin, fluorocarbon resin, urea resin, phenol resin, epoxy resin, polyamide resin, acrylic resin, methacrylic resin, and derivatives thereof. The preferable one is polyurethane resin, especially segmented polyurethane resin.

The segmented polyurethane resin is prepared synthetically from three components: a polyol, a diisocyanate, and a chain elongation agent and thus has elastomeric characteristics according to a block polymer structure having hard segments and soft segments within molecular. Therefore, the elasticity achieved by using this segmented polyurethane resin was expected to exhibit an effect of attenuating the stress generated at interface between subcutaneous tissue and the cuff when the patient, the catheter, or the cannula moves or when skin around the portion of the cuff insertion is moved during the disinfection process.

The cuff of the present invention may comprise a layer having the said specific porous three-dimensional network structure as a first layer and a second layer, laminated on the first layer, having a structure different from that of the first layer. The second layer may be a fiber aggregation, a flexible film, or a porous three-dimensional network structure of which the average pore diameter and the apparent density are different from those of the porous three-dimensional network structure of the first layer.

The fiber aggregation may be, for example, unwoven fabric or woven fabric, of which thickness is from 0.1 to 100 mm, preferably from 0.1 to 50 mm, more preferably from 0.1 to 10 mm, particularly preferably from 0.1 to 5 mm. The thickness in this range is preferable because well flexibility is maintained when laminated on the porous three-dimensional network structure and robust bonding with subcutaneous tissue is obtained.

Porosity of the unwoven fabric or woven fabric is preferably in the range of from 100 to 5000 cc/cm²/min in view of flexibility, connecting strength with subcutaneous tissue, and the like. It should be noted that “porosity” used here is a value measured according to JIS L 1004 and is sometimes called as air permeability or ventilation volume.

The fiber aggregation may be made of synthetic resin composing of one or more selected from a group composing of polyurethane resin, polyamide resin, polylactide resin, polyolefin resin, polyester resin, fluorocarbon resin, acrylic resin, methacrylic resin, and derivatives thereof. The fiber aggregation may also be made of naturally-occurring fibers composing of one or more selected from a group composing of fibroin, chitin, chitosan, and cellulose, and derivatives thereof. In addition, mixture of synthetic fibers and naturally-occurring fibers may also be used.

The flexible film may be a thermoplastic resin film, especially a film made of one or more selected from a group composing of polyurethane resin, polyamide resin, polylactide resin, polyolefin resin, polyester resin, fluorocarbon resin, urea resin, phenol resin, epoxy resin, polyimide resin, acrylic resin, methacrylic resin, and derivatives thereof. The flexible film is preferably a film made of one or more selected from a group composing of polyester resin, fluorocarbon resin, polyurethane resin, acrylic resin, vinyl chloride, fluorocarbon resin, and silicone resin.

Thickness of the flexible film ranging from 0.1 to 500 mm makes a cuff which is advantageous in view of flexibility and physical strength. The thickness of the flexible film is preferably from 0.1 to 100 mm, more preferably from 0.1 mm to 50 mm, furthermore preferably from 0.1 mm to 10 mm.

The flexible film may be not only a solid film but also a porous film or a foamed film. By laminating a solid flexible film, a cuff which has excellent antibacterial property and is therefore advantageous in transmission maintenance is obtained.

When a porous three-dimensional network structure of which the average pore diameter and the apparent density are different from that of the porous three-dimensional network structure of the first layer is used as the second layer, the second layer may be a porous three-dimensional network structure having an average pore diameter of from 0.1 to 200 μm and an apparent density of from 0.01 to 1.0 g/cm³. The thickness of the porous three-dimensional network structure of the second layer preferably ranges from 0.2 mm to 20 mm.

As for the method of laminating the second layer onto the porous three-dimensional network structure, when the second layer is a fiber aggregation, a flexible film, or a porous three-dimensional network structure of which the average pore diameter and/or the apparent density are different from those of the porous three-dimensional network structure of the first layer, a bonding method using adhesives, particularly, a method of inserting a hot-melt unwoven fabric between the first layer and the second layer and pressing them under heating condition may be employed. The hot-melt unwoven fabric may be a polyamide type hot-melt adhesive sheet such as PA1001 available from Nitto Boseki Co., Ltd. or the like. Alternatives are a method of bonding by melting an outer layer of a contact surface with a solvent, a method of bonding by melting an outer layer with heating, and a method using ultrasonic sound or high frequency wave. Further, during the preparation of the first layer, the fiber aggregation or the flexible film may be laminated on the polymer dope. In this manner, the second layer can be laminated and formed in a continuous fashion.

The second layer may be formed of two or more of the fiber aggregation, the flexible film, and the porous three-dimensional network structure. The cuff may be a three layer structure in which another porous three-dimensional network structure same as the first layer may also be laminated via the second layer.

In the porous three-dimensional network structure of the cuff of the present invention, one or more selected from a group composing of collagen Type I, collagen Type II, collagen Type III, collagen Type IV, atelocollagen, fibronectin, gelatin, hyaluronic acid, heparin, keratin acid, chondroitin, chondroitin sulfate, condroitin sulfate B, elastin, heparan sulfate, laminin, thrombospondin, hydronectin, osteonectin, entactin, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone may be held. Further in the porous three-dimensional network structure, one or more selected from a group composing of platelet-derived growth factor, epidermal growth factor, transforming growth factor-α, insulin-like growth factor, insulin-like growth factor binding proteins, hepatocyte growth factor, vascular endothelial proliferation growth factor, angiopoietin, nerve growth factor, brain-derived neurotrophic factor, ciliary neurotrophic factor, transforming growth factor-β, latent form transforming growth factor-β, activin, bone plasma proteins, fibrocyte growth factor, tumor growth factor-β, diploid fibrocyte growth factor, heparin-binding epidermal growth factor-like growth factor, schwannoma-derived growth factor, anfillegrin, betacellulin, epillegrin, lymphotoxin, erythropoietin, tumor necrosis factor-α, interleukin-1β, interleukin-6, interleukin-8, interleukin-17, interferon, antivirotic, antimicrobial agent, and antibacterial agent may be held. Furthermore in the porous three-dimensional network structure, cells of one or more kinds selected from a group composing of embryo-stem cell (which may be dividing cell), vascular endothelial cell, mesodermal cell, smooth muscle cell, peripheral vessel cell, and mesothelial cell may be attached.

The cuff of the present invention enables its skeleton made of the thermoplastic resin or the thermosetting resin constructing the porous three-dimensional network structure to be provided with fine pores. These fine pores make the skeleton to have complex irregular surface not smooth surface. The irregular surface is effective in holding collagen and cell growth factor, resulting in increased cell engraftment. These fine pores are outside the concept of calculating the average pore diameter of the porous three-dimensional network structure as employed in the present invention.

Hereinafter, an example of method for preparing the porous three-dimensional network structure made of thermoplastic polyurethane resin constructing the cuff of the present invention will be described, but the preparing method of the cuff of the present invention is not limited to the following method at all.

To prepare a porous three-dimensional network structure made of a thermoplastic polyurethane resin, first a polymer dope is prepared by mixing a polyurethane resin, a water-soluble polymer compound, as will be described later, as a pore forming agent, and an organic solvent as a good solvent for the polyurethane resin. Specifically, after the polyurethane resin is mixed into the organic solvent to have a homogeneous solution, a water-soluble polymer compound is mixed and dissolved into this homogeneous solution. Examples of the organic solvent include N,N-dimethylformamide, N-methyl-2-pyrrolidinone, and tetrahydrofuran. However, the organic solvent to be used is not limited thereto and may be any organic solvent capable of solving the thermoplastic polyurethane resin. In addition, the polyurethane resin may be dissolved by heat with reduced amount of organic solvent or without organic solvent and the pore forming agent may be mixed to the dissolved polyurethane resin.

Examples of the water-soluble polymer compound as pore forming agent include polyethylene glycol, polypropylene glycol, polyvinyl alcohol, polyvinyl pyrrolidone, alginic acid, carboxymethyl cellulose, hydroxypropyl cellulose, methyl cellulose, and ethyl cellulose. However, the water-soluble polymer compound to be used is not limited thereto and may be any water-soluble polymer compound capable of being homogeneously dispersed with the thermoplastic resin to form a polymer dope. In addition, depending on the kind of the thermoplastic resin, the polymer compound is not limited to water-soluble ones. For example, lipophilic compounds such as phthalate ester and paraffin, and inorganic salts such as lithium chloride and calcium carbonate may be used. It is also available to use crystal-nucleation agent for polymer so as to generate secondary particles during coagulation, that is, to encourage skeletal formation of porous body.

The polymer dope prepared from the thermoplastic polyurethane resin, the organic solvent, and water-soluble polymer compound is dipped in coagulation bath containing a poor solvent of thermoplastic polyurethane resin so as to extract and remove organic solvent and water-soluble polymer compound in the coagulation bath. By removing a part or all of organic solvent and water-soluble polymer compound, a porous three-dimensional structural material of polyurethane resin is obtained. Examples of the poor solvent used herein include water, lower alcohol, and low carbon number ketones. The coagulated polyurethane resin is finally washed with water or the like to remove remaining organic solvent and pore forming agent.

Hereinafter, a preferred embodiment of the biological implant covering member of the present invention will be described.

The biological implant covering member of the present invention is composed of a three-dimensional network structure having well communication property made of thermoplastic resin or thermosetting resin. The three-dimensional network structure is a porous three-dimensional network structure having an average pore diameter of from 100 to 1000 μm and apparent density of from 0.01 to 0.5 g/cm³. In the cutting surfaces in the depth direction, the surfaces may be entirely similar or one side surface is different from the other side surface. The average pore diameter and/or the apparent density may partially vary. For example, the average pore diameter may vary gradually from the one side surface to the other side surface, that is, the three-dimensional network structure may have anisotropy. The three-dimensional network structure may be provided, in the contact surface with native tissues, with pores having a large pore diameter which is extremely larger than the average pore diameter. It is preferable that these pores are pores having a pore diameter in the range of from 500 to 2000 μm. These pores existing near the outer layer on the side of native tissues facilitate extracellular matrix such as collagen to homogeneously penetrate deep parts and effectively act on infiltration of cells from tissues and neovascularization of capillary vessels. It should be noted that such large diameter pores are outside the concept of calculating the average pore diameter of the porous three-dimensional network structure as employed in the present invention.

As for the porous three-dimensional network structure, the average pore diameter is from 100 to 1000 μm and the apparent density is from 0.01 to 0.5 g/cm³. The average pore diameter is preferably from 200 to 600 μm, more preferably from 200 to 50 μm. The apparent density in the range of from 0.01 to 0.5 g/cm³ can provide well cell engraftment, excellent physical strength, and elastic characteristics similar to subcutaneous tissues when cells infiltrate, are sufficiently grown and tightly interconnected. The apparent density is preferably from 0.05 to 0.3 g/cm³, more preferably from 0.05 to 0.2 g/cm³.

As for the distribution of pore diameter with the same average pore diameter, higher contribution ratio of pores of 150-400 μm diameter that is important in allowing cell infiltrate is better. The contribution ratio of pores of 150-400 μm diameter is 10% or more, preferably 20% or more, more preferably 30% or more, particularly preferably 40% or more, especially preferably 50% or more. Such contribution ratio is preferable because it enables cells to easily invade and allows the invaded cell to easily adhere and grow.

The contribution ratio of pores of 150-400 μm diameter in the average pore diameter of the porous three-dimensional network structure denotes a ratio of the number of pores of 150-400 μm diameter relative to the number of all pores in a measuring method of average pore diameter in Example 4 described later.

The porous three-dimensional network structure having the average pore diameter, the apparent density, and the distribution of pore diameters as mentioned above allows easy infiltration of cells into pores and allows easy adhesion and growth of cells onto the porous three-dimensional network structure, thereby constructing capillary vessels. Therefore, with this porous three-dimensional network structure, an excellent biological implant covering member which can provide a robust bonding to subcutaneous tissue at a portion where it is inserted can be obtained.

The porous three-dimensional structure may have a thickness ranging from 0.5 mm to 500 mm. The thickness is preferably from 0.5 to 100 mm, more preferably from 0.5 to 50 mm, particularly preferably from 0.5 to 10 mm, especially preferably from 0.5 to 5 mm. Such a thickness as mentioned provides a high level of satisfaction in physical strength required as a biological implant covering member, infiltrate of cells, organization, and bonding with subcutaneous tissue.

The thermoplastic resin or thermosetting resin composing the porous three-dimensional network structure may be one or more of polyurethane resin, polyamide resin, polylactide resin, polymalate resin, polyglycolate resin, polyolefin resin, polyester resin, fluorocarbon resin, urea resin, phenol resin, epoxy resin, polyimide resin, acrylic resin, methacrylic resin, and derivatives thereof. The preferable one is polyurethane resin, especially segmented polyurethane resin.

The segmented polyurethane resin is prepared synthetically from three components: a polyol, a diisocyanate, and a chain elongation agent and thus has elastomeric characteristics according to a so-called block polymer structure having hard segments and soft segments within molecular. Therefore, the elasticity achieved by using this segmented polyurethane resin was expected to exhibit an effect of attenuating the stress generated at interface between subcutaneous tissue and the biological implantation member.

The biological implant covering member of the present invention may comprise a layer having the said specific porous three-dimensional network structure as a first layer and a second layer, laminated on the first layer, having a structure different from that of the first layer. The second layer may be a porous three-dimensional network structure of which the average pore diameter and the apparent density are different from those of the porous three-dimensional network structure as the first layer.

In the porous three-dimensional network structure of the biological implant covering member, one or more selected from a group composing of collagen Type I, collagen Type II, collagen Type III, collagen Type IV, atelocollagen, fibronectin, gelatin, hyaluronic acid, heparin, keratin acid, chondroitin, chondroitin sulfate, condroitin sulfate B, elastin, heparan sulfate, laminin, thrombospondin, hydronectin, osteonectin, entactin, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone may be held. Further in the porous three-dimensional network structure, one or more selected from a group composing of platelet-derived growth factor, epidermal growth factor, transforming growth factor-α, insulin-like growth factor, insulin-like growth factor binding proteins, hepatocyte growth factor, vascular endothelial proliferation growth factor, angiopoietin, nerve growth factor, brain-derived neurotrophic factor, ciliary neurotrophic factor, transforming growth factor-β, latent form transforming growth factor-β, activin, bone plasma proteins, fibrocyte growth factor, tumor growth factor-β, diploid fibrocyte growth factor, heparin-binding epidermal growth factor-like growth factor, schwannoma-derived growth factor, anfillegrin, betacellulin, epillegrin, lymphotoxin, erythropoietin, tumor necrosis factor-α, interleukin-1β, interleukin-6, interleukin-8, interleukin-17, interferon, antivirotic, antimicrobial agent, and antibacterial agent may be held. Furthermore in the porous three-dimensional network structure, cells of one or more kind selected from a group composing of embryo-stem cell (which may be dividing cells), vascular endothelial cell, mesodermal cell, smooth muscle cell, peripheral vessel cell, and mesothelial cell may be attached.

The biological implant covering member of the present invention enables its skeleton made of the thermoplastic resin or the thermosetting resin constructing the porous three-dimensional network structure to be provided with fine pores. These fine pores make the skeleton to have complex irregular surface not smooth surface. The irregular surface is effective in holding collagen and cell growth factor, resulting in increased cell engraftment. These fine pores are outside the concept of calculating the average pore diameter of the porous three-dimensional network structure as employed in the present invention.

Hereinafter, an example of method for preparing the porous three-dimensional network structure made of thermoplastic polyurethane resin constructing the biological implant covering member of the present invention will be described, but the preparing method of the biological implant covering member of the present invention is not limited to the following method at all.

To prepare a porous three-dimensional network structure made of a thermoplastic polyurethane resin, first a polymer dope is prepared by mixing a polyurethane resin, a water-soluble polymer compound, as will be described later, as a pore forming agent, and an organic solvent as a good solvent for the polyurethane resin. Specifically, after the polyurethane resin is mixed into the organic solvent to have a homogeneous solution, a water-soluble polymer compound is mixed and dissolved into this homogeneous solution. Examples of the organic solvent include N,N-dimethylformamide, N-methyl-2-pyrrolidinone, and tetrahydrofuran. However, the organic solvent to be used is not limited thereto and may be any organic solvent capable of solving the thermoplastic polyurethane resin. In addition, the polyurethane resin may be dissolved by heat with reduced amount of organic solvent or without organic solvent and the pore forming agent may be mixed to the dissolved polyurethane resin.

Examples of the water-soluble polymer compound as pore forming agent include polyethylene glycol, polypropylene glycol, polyvinyl alcohol, polyvinyl pyrrolidone, alginic acid, carboxymethyl cellulose, hydroxypropyl cellulose, methyl cellulose, and ethyl cellulose. However, the water-soluble polymer compound to be used is not limited thereto and may be any water-soluble polymer compound capable of being homogeneously dispersed with the thermoplastic resin to form a polymer dope. In addition, depending on the kind of the thermoplastic resin, the polymer compound is not limited to water-soluble ones. For example, lipophilic compounds such as phthalate ester and paraffin, and inorganic salts such as lithium chloride and calcium carbonate may be used. It is also available to use crystal-nucleation agent for polymer so as to generate secondary particles during coagulation, that is, to encourage skeletal formation of porous body.

The polymer dope prepared from the thermoplastic polyurethane resin, the organic solvent, and water-soluble polymer compound is dipped in coagulation bath containing a poor solvent of thermoplastic polyurethane resin so as to extract and remove organic solvent and water-soluble polymer compound in the coagulation bath. By eliminating a part or all of organic solvent and water-soluble polymer compound, a porous three-dimensional structural material of polyurethane resin is obtained. Examples of the poor solvent used herein include water, lower alcohol, and low carbon number ketones. The coagulated polyurethane resin is finally washed with water or the like to remove remaining organic solvent and pore forming agent.

As described above, the biological implant covering member of the present invention allows easy infiltration of cells from native tissues, easy engraftment of cells, and organization, thereby obtaining robust bonding with native tissues and therefore protecting a living body from adverse effect which may occur due to the insertion of a biological implantation member into the living body.

Hereinafter, the cuff and the biological implant covering member composing the surface thereof will be described in detail with reference to the following examples, but the present invention is not limited by the following examples at all without departing from the scope of the invention.

### EXAMPLE 4

A thermoplastic polyurethane resin (MIRACTRAN E980PNAT available from Nippon Miractran Co., Ltd.) was dissolved into N-methyl-2-pyrrolidinone (reagent for peptide synthesis, NMP available from Kanto Kagaku) by using a dissolver (about 2000 rpm) at room temperature to obtain 7.5% solution (weight/weight). 1.0 kg of this NMP solution was measured and entered into a planetary mixer (PLM-2 type, capacity 2.0 liters, available from Inoue Mfg., Inc.) and was mixed with methylcellulose (reagent, 50 cp grade, available from Kanto Kagaku) of an amount corresponding to half the amount of polyurethane resin at a temperature of 40° C. for 20 minutes. With the agitation being continued, the defoaming was conducted by reducing the pressure to 20 mmHg (2.7 kPa) for 10 minutes, thereby obtaining polymer dope.

Two Teflon plates of 3 mm in thickness and of 150 mm×150 mm were prepared and each inner section of 140 mm×140 mm was punched in each plate, thereby forming two square frames. The two square frames were superposed and a chemical experimental paper filter (Qualitative filter paper No. 1, available from Toyo Roshi Kaisha, Ltd.) was inserted and fixed therebetween so as to form a Teflon frame unit. The said polymer dope was filled into the frame unit and wiped by glass bar in order to drain excess dope off by using a glass bar. After that, a chemical experimental paper filter (Qualitative filter paper No. 1, available from Toyo Roshi Kaisha, Ltd.) was put on as a cover sheet and fixed to the frame unit to hold the filled polymer dope. The frame unit was entered into methanol under refluxing condition. The refluxing was continued for 72 hours to extract and remove NMP solution through the chemical experimental paper filters on both sides of the frame unit, whereby the polyurethane resin was coagulated. During this, the methanol was replaced with new one as needed with keeping the refluxing condition.

After 72 hours, the solidificated polyurethane resin was removed from the Teflon frame unit and was washed in purified water of the Japanese Pharmacopoeia for 72 hours to extract and remove methylcellulose, methanol, and remaining NMP. The water for washing was replaced with new one as needed. The washed content was depressurized (20 mmHg) at room temperature for 24 hours and dried, thereby obtaining a porous three-dimensional network structural material made of thermoplastic polyurethane resin. The porous three-dimensional network structural material was a biological implant covering member of the present invention.

Fabric velour made of polyester of 140 mm×140 mm (Bobeiky Double Velour Fabric, having a porosity of 3800 cc/cm²/min and a thickness of 1.5 mm, available from Bird Company) was impregnated with tetrahydrofuran (reagent of superfine quality available from Kanto Kagaku) and was wrung by two rollers to have impregnated amount of 0.104±0.002 g/cm². The said porous three-dimensional network structural material (the biological implant covering member) was superposed onto the fabric velour impregnated with tetrahydrofuran and was pressed by a load of 1.0 kg/cm², thereby obtaining a cuff of the present invention.

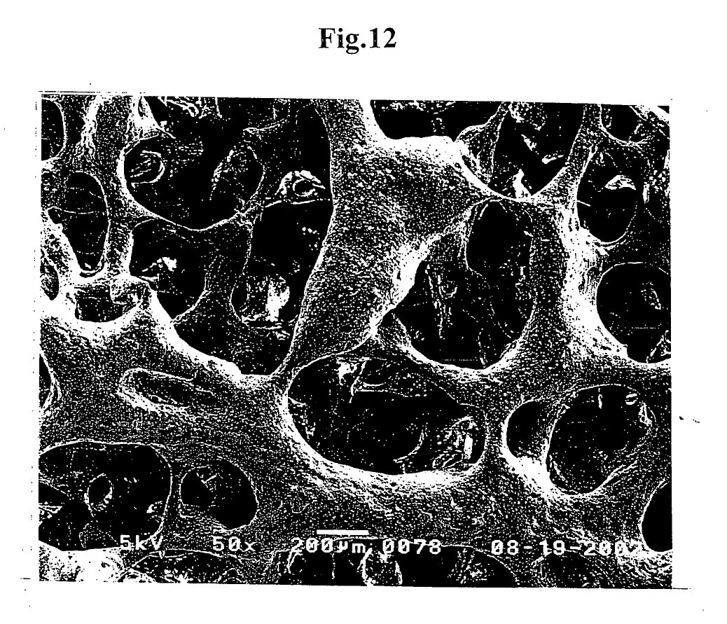

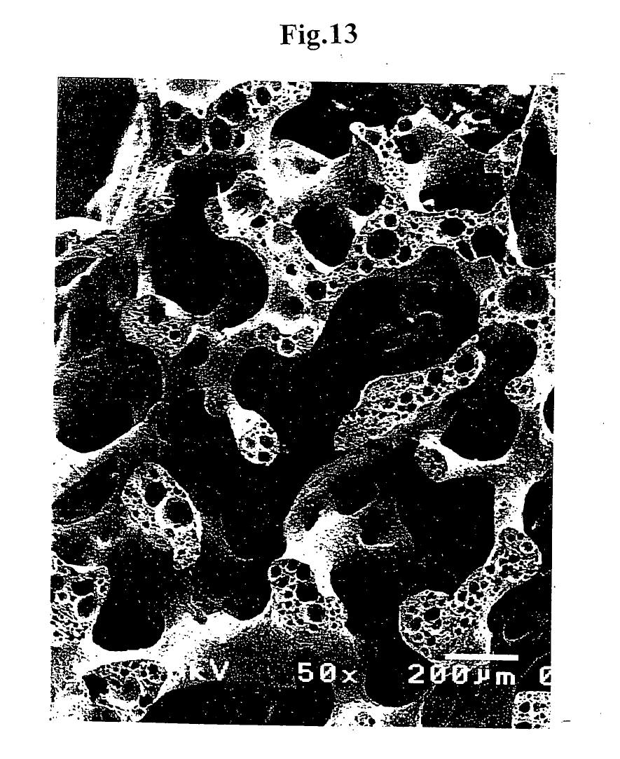

FIG. 12 and FIG. 13 are pictures of the biological implant covering member on the surface of the cuff taken by a scanning electron microscope (SM200 available from TOPCON Corporation). From these pictures, it is found that the biological implant covering member on the surface of the obtained cuff is a porous three-dimensional network structure of 350 μm in pore diameter.

As for the porous three-dimensional network structure portion (i.e. the biological implant covering member) of 2.3 mm in thickness of the obtained cuff, the average pore diameter and the apparent density were measured in the following methods. The results are shown in Table 1. In the measurements of the average pore diameter and the apparent density, specimens were cut by using a twin bladed razor (HighStainless available FEATHER Safety Razor Co., Ltd) at room temperature.

[Measurement of Average Pore Diameter]

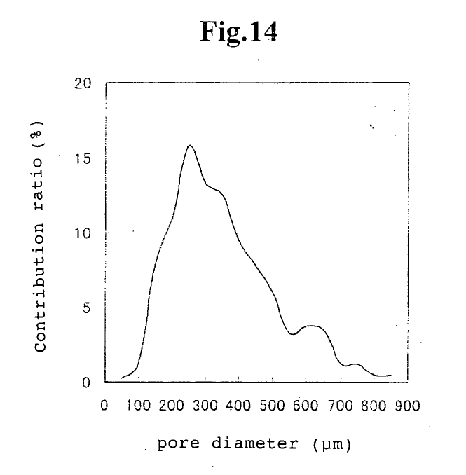

By using a picture of a plane (cutting surface) of specimen, cut by the twin bladed razor, taken by an electron microscope (SM200 available from TOPCON Corporation), image processing was conducted to take respective pores on the same plane as figures surrounded by skeleton of three-dimensional network structure (using LUXEX AP available from NIRECO Corporation as an image processing unit and LE N50 available from SONY Corporation as a CCD camera for taking images) and the areas of the respective figures were measured. The areas were converted to areas of real circles. The diameters of the corresponding circles were obtained as the pore diameters. Measurement was conducted only for through pores on the same plane in disregard for micropores bored in the porous skeleton. At the same time, the distribution of pore diameters regarding all measured pores was measured and shown in FIG. 14. The contribution ratio of pores of 150-400 μm diameter was obtained from the measurement result of pore diameter distribution.

[Measurement of Apparent Density]

The three-dimensional network structure prepared in Example 4 before lamination of a second layer was cut into a cubic specimen of about 10 mm×10 mm×3 mm by the twin bladed razor. The volume of the specimen was obtained from dimensions measured by a projector (V-12, Nikon). The apparent density was obtained by dividing the weight by the volume.

TABLE 1

| | Average | Contribution | Apparent | |
|---|---|---|---|---|
| | pore | ratio of 150-400 | density | Thickness |
| | diameter (μm) | μm pores (%) | (g/cm³) | (mm) |
| Porous three- | 329 ± 160 | 62.2 | 0.117 ± 0.008 | 2.3 |
| dimensional network | | | | |
| structure as first | | | | |
| layer | | | | |

It is apparent from Table 1 that the porous three-dimensional network structure as the first layer is a porous three-dimensional network structure mainly having pores effective in cell adhesion.

### EXAMPLE 5

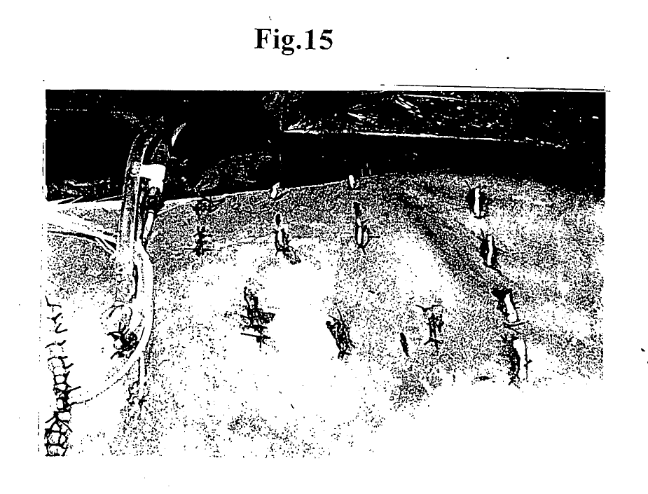

An adult goat (female, weight 54 kg) was prepared as an analyte and a portion of shaved skin from left thoracic part to abdominal part was used as a test substance. During operation, the analyte was rapidly inserted with an endotracheal tube in a left supine position of in an ordinal technical manner and was maintained under general anesthesia by isoflurane. The surface of a portion including the thoracic part and abdominal part was sterilized with Isodine. After that, the surface was incised 20 mm and a half of the specimen of the cuff prepared in Example 4 was implanted and fixed by suturing subcutaneous tissue (FIG. 15). The cuff was cut into a specimen of 10 mm×10 mm and was subjected to ethylene oxide gas sterilization. After the operation, the test substance was sterilized with acid water or Isodine twice a day. The analyte drunk water freely and was supplied with a suitable amount (about 1 kg) of haycubes as fodder five times a day. After two weeks from the operation, the specimens previously implanted and peripheral tissues were removed from the analyte under general anesthesia. The specimen and the peripheral tissue were engrafted tightly so that exfoliation therebetween was difficult, and there were no evidences of infection, inflammation, and the like in peripheries.

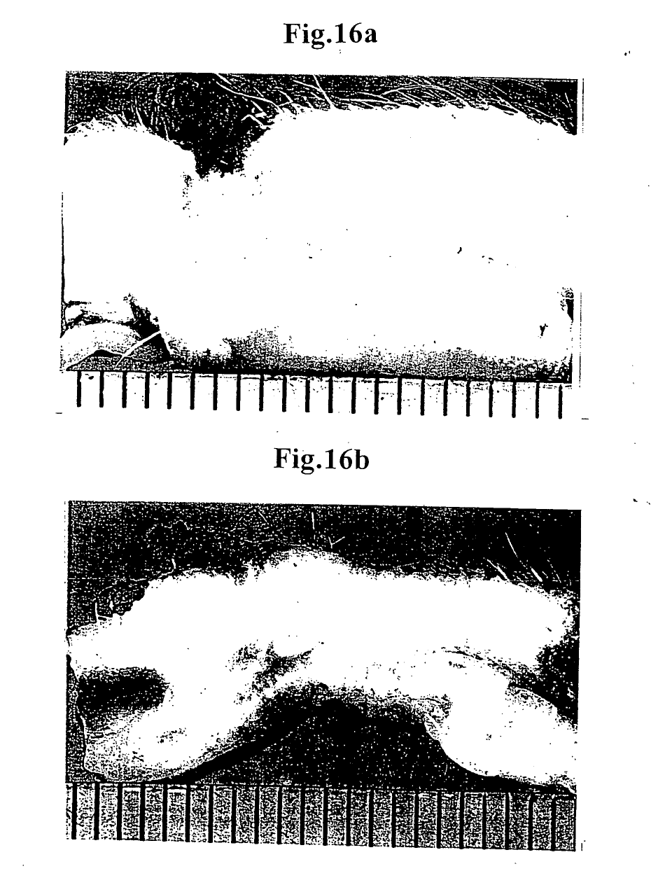

FIG. 16a is a picture of the surface of the cuff (i.e. the biological implant covering member) showing an engrafted portion enlarged by a loupe. A poorly-demarcated milk-white layer, indicated by an arrow in FIG. 16a, extended to the inside of the cuff and the inside of the cuff was filled with transparent tissues. From this, it was recognized that granulation tissues were infiltrated.

FIG. 16b is a picture enlarged by a loupe in case that the same test was conducted using a woven fabric (fabric velour made of polyester (Bobeiky Double Velour Fabric available from Bird Company) used in Example 4) alone. A milk-white layer was infiltrated along the surface of the fabric only in the depth direction not near the outer surface, that is, the downgrowth phenomenon was confirmed.

Unlike this, in case of the cuff of the present invention, it was found that the milk-white layer continuously extended to near the outer skin so that the downgrowth phenomenon was inhibited.

After the tests, the extracted specimens were fixed promptly by 10% neutral buffered formalin and HE stained samples were prepared in ordinary method. The samples were observed by an optical microscope. As a result, it is recognized that granulation tissues mainly comprising extracellular matrix such as fibrocyte, macrophage, and collagen fibril extending from the surrounding tissues were infiltrated and vascularization was observed.

It was recognized from the samples obtained by the same procedure after four weeks that many granulation tissues extended and further grown bonding tissues were formed on the embedded specimens. That is, it was observed that the organization further advanced.

As described above, the cuff of the present invention enables further organization by the infiltration of living cells into the porous three-dimensional network structure and ensures separation of a wounded portion from the outside, thereby protecting against exacerbation factors such as bacterial infection on healing.

As described above in detail, the cuff of the present invention allows easy infiltration of cells from living subcutaneous tissues, easy engraftment of cells, and neovascularization of capillary vessels so as to obtain robust bonding with subcutaneous tissues. As a result, separation of a wounded portion from the outside is ensured, thereby blocking exacerbation factors such as bacterial infection on healing and inhibiting progression of downgrowth. That is, the invention provides a cuff with none or little infection trouble such as tunnel infection.

The cuff of the present invention as mentioned above can be suitably used for blood circulation method by ventricular assist device, which is a treatment implanting a cannula or catheter subcutaneously, peritoneal dialysis therapy, central venous nutrition method, and for the implant part of living skin for such as transcannula DDS, transcatheter DDS, or the like.

1. A scaffold material for tissue engineering made of thermoplastic resin which forms a porous three-dimensional network structure having communication property, wherein the porous three-dimensional network structure has an average pore diameter of from 100 to 650 μm and an apparent density of from 0.01 to 0.5 g/cm³.

2. A scaffold material for tissue engineering as claimed in claim 1, wherein the average pore diameter of said porous three-dimensional network structure is from 100 to 400 μm and the apparent density thereof is from 0.01 to 0.5 g/cm³.

3. A scaffold material for tissue engineering as claimed in claim 2, wherein the average pore diameter of said porous three-dimensional network structure is from 100 to 300 μm.

4. A scaffold material for tissue engineering as claimed in any one of claim 1 through 3, wherein the apparent density of said porous three-dimensional network structure is from 0.01 to 0.2 g/cm³.

5. A scaffold material for tissue engineering as claimed in claim 4, wherein the apparent density of said porous three-dimensional network structure is from 0.01 to 0.1 g/cm³.

6. A scaffold material for tissue engineering as claimed claim 1, wherein the contribution ratio of pores of 150-300 μm diameter in the average pore diameter of the porous three-dimensional network structure is 10% or more.

7. A scaffold material for tissue engineering as claimed in claim 6, wherein the contribution ratio of pores of 150-300 μm diameter in the average pore diameter of the porous three-dimensional network structure is 20% or more.

8. A scaffold material for tissue engineering as claimed in claim 7, wherein the contribution ratio of pores of 150-300 μm diameter in the average pore diameter of the porous three-dimensional network structure is 30% or more.

9. A scaffold material for tissue engineering as claimed in claim 8, wherein the contribution ratio of pores of 150-300 μm diameter in the average pore diameter of the porous three-dimensional network structure is 40% or more.

10. A scaffold material for tissue engineering as claimed in claim 9, wherein the contribution ratio of pores of 150-300 μm diameter in the average pore diameter of the porous three-dimensional network structure is 50% or more.

11. A scaffold material for tissue engineering as claimed in claim 1, wherein said thermoplastic resin is one or more selected from a group composing of polyurethane resin, polyamide resin, polylactide resin, polyolefin resin, polyester resin, fluorocarbon resin, acrylic resin, methacrylic resin, and derivatives thereof.

12. A scaffold material for tissue engineering as claimed in claim 11, wherein the thermoplastic resin is polyurethane resin.

13. A scaffold material for tissue engineering as claimed in claim 12, wherein the polyurethane resin is segmented polyurethane resin.

14. A scaffold material for tissue engineering as claimed in claim 1, wherein one or more selected from a group composing of collagen Type I, collagen Type II, collagen Type III, collagen Type IV, atelocollagen, fibronectin, gelatin, hyaluronic acid, heparin, keratin acid, chondroitin, chondroitin sulfate, condroitin sulfate B, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone is held in the porous three-dimensional network structure.

15. A scaffold material for tissue engineering as claimed in claim 14, wherein one or more selected from a group composing of fibrocyte growth factor, interleukin-1, tumor growth factor-β, epidermal growth factor, and diploidic fibrocyte growth factor is further held in the porous three-dimensional network structure.

16. A scaffold material for tissue engineering as claimed in claim 15, wherein cells are adhered on the porous three-dimensional network structure.

17. A scaffold material for tissue engineering as claimed in claim 16, wherein said cells are cells of one or more kinds selected from a group composing of embryo-Nakayama stem cell, vascular endothelial cell, mesodermal cell, smooth muscle cell, peripheral vessel cell, and mesothelial cell.

18. A scaffold material for tissue engineering as claimed in claim 17, wherein said embryo-stem cell is dividing cell.

19. A scaffold material for tissue engineering as claimed in claim 1, wherein the scaffold takes the form of a tubular body.

20. A scaffold material for tissue engineering as claimed in claim 19, wherein the tubular body is from 0.3 to 15 mm in inner diameter and from 0.4 to 20 mm in outer diameter.

21. A scaffold material for tissue engineering as claimed in claim 20, wherein the tubular body is from 0.3 to 10 mm in inner diameter and from 0.4 to 15 mm in outer diameter.

22. A scaffold material for tissue engineering as claimed in claim 21, wherein the tubular body is from 0.3 to 6 mm in inner diameter and from 0.4 to 10 mm in outer diameter.

23. A scaffold material for tissue engineering as claimed in claim 22, wherein the tubular body is from 0.3 to 2.5 mm in inner diameter and from 0.4 to 10 mm in outer diameter.

24. A scaffold material for tissue engineering as claimed in claim 23, wherein the tubular body is from 0.3 to 1.5 mm in inner diameter and from 0.4 to 10 mm in outer diameter.

25. An artificial blood vessel being composed of A scaffold material as claimed in claim 1.

26. An artificial blood vessel as claimed in claim 25, wherein the outside of the scaffold is covered by another tubular body.

27. An artificial blood vessel as claimed in claim 26, wherein the tubular body covering the outside of the scaffold is a tube made of one or more selected from a group composing of chitosan, polylactide resin, polyester resin, polyamide resin, polyurethane resin, fibronectin, gelatin, hyaluronic acid, keratin acid, chondroitin, chondroitin sulfate, condroitin sulfate B, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone, cross-linked collagen, and fibroin.

28. A cuff comprising a porous three-dimensional network structure which is made of a substrate resin composed of thermoplastic resin or thermosetting resin and has communication property, wherein the porous three-dimensional network structure has an average pore diameter of from 100 to 1000 μm and apparent density of from 0.01 to 0.5 g/cm³.

29. A cuff as claimed in claim 28, wherein the average pore diameter of said porous three-dimensional network structure is from 200 to 600 μm and the apparent density is from 0.01 to 0.5 g/cm³.

30. A cuff as claimed in claim 29, wherein the average pore diameter of said porous three-dimensional network structure is from 200 to 500 μm and the apparent density is from 0.01 to 0.5 g/cm³.

31. A cuff as claimed in any one of claims 28 through 30, wherein the apparent density of said porous three-dimensional network structure is from 0.05 to 0.3 g/cm³.

32. A cuff as claimed in claim 31, wherein the apparent density of said porous three-dimensional network structure is from 0.05 to 0.2 g/cm³.

33. A cuff as claimed in claim 28, wherein the contribution ratio of pores of 150-400 μm diameter in the average pore diameter of the porous three-dimensional network structure is 10% or more.

34. A cuff as claimed in claim 33, wherein the contribution ratio of pores of 150-400 μm diameter in the average pore diameter of the porous three-dimensional network structure is 20% or more.

35. A cuff as claimed in claim 34, wherein the contribution ratio of pores of 150-400 μm diameter in the average pore diameter of the porous three-dimensional network structure is 30% or more.

36. A cuff as claimed in claim 35, wherein the contribution ratio of pores of 150-400 μm diameter in the average pore diameter of the porous three-dimensional network structure is 40% or more.

37. A cuff as claimed in claim 36, wherein the contribution ratio of pores of 150-400 μm diameter in the average pore diameter of the porous three-dimensional network structure is 50% or more.

38. A cuff as claimed in any one of claims 28 through 37, wherein the thickness of the porous three-dimensional network structure is from 0.2 to 500 mm.

39. A cuff as claimed in claim 38, wherein the thickness of the porous three-dimensional network structure is from 0.2 to 100 mm.

40. A cuff as claimed in claim 39, wherein the thickness of the porous three-dimensional network structure is from 0.2 to 50 mm.

41. A cuff as claimed in claim 40, wherein the thickness of the porous three-dimensional network structure is from 0.2 to 10 mm.

42. A cuff as claimed in claim 41, wherein the thickness of the porous three-dimensional network structure is from 0.2 to 5 mm.

43. A cuff as claimed in claim 28, wherein said substrate resin is one or more selected from a group composing of polyurethane resin, polyamide resin, polylactide resin, polyolefin resin, polyester resin, fluorocarbon resin, urea resin, phenol resin, epoxy resin, polyamide resin, acrylic resin, methacrylic resin, and derivatives thereof.

44. A cuff as claimed in claim 43, wherein the substrate resin is polyurethane resin.

45. A cuff as claimed in claim 44, wherein the polyurethane resin is segmented polyurethane resin.

46. A cuff as claimed in claim 28, wherein the cuff is a lamination of a first layer composed of the porous three-dimensional network structure and a second layer different from the first layer.

47. A cuff as claimed in claim 46, wherein the second layer is one or more selected from a group composing of a fiber aggregation, a flexible film, and a porous three-dimensional network structure of which the average pore diameter and the apparent density are different from those of the porous three-dimensional network structure of the first layer.

48. A cuff as claimed in claim 47, wherein the fiber aggregation is unwoven fabric or woven fabric.

49. A cuff as claimed in claim 48, wherein the thickness of the unwoven fabric or woven fabric is 0.1-100 mm.

50. A cuff as claimed in claim 49, wherein the thickness of the unwoven fabric or woven fabric is 0.1-50 mm.

51. A cuff as claimed in claim 50, wherein the thickness of the unwoven fabric or woven fabric is 0.1-10.0 mm.

52. A cuff as claimed in claim 51, wherein the thickness of the unwoven fabric or woven fabric is 0.1-5.0 mm.

53. A cuff as claimed in any one of claims 48 through 52, wherein the porosity of the unwoven fabric or woven fabric is from 100 to 5000 cc/cm²/min.

54. A cuff as claimed in claim 46, wherein the fiber aggregation is made of one or more selected from a group composing of polyurethane resin, polyamide resin, polylactide resin, polyolefin resin, polyester resin, fluorocarbon resin, acrylic resin, methacrylic resin, and derivatives thereof.

55. A cuff as claimed in claim 46, wherein the fiber aggregation is made of one or more selected from a group composing of fibroin, chitin, chitosan, and cellulose, and derivatives thereof.

56. A cuff as claimed in claim 46, wherein the flexible film is a thermoplastic resin film.

57. A cuff as claimed in claim 56, wherein the thermoplastic resin is one or more selected from a group composing of polyurethane resin, polyamide resin, polylactide resin, polyolefin resin, polyester resin, fluorocarbon resin, urea resin, phenol resin, epoxy resin, polyimide resin, silicone resin, acrylic resin, methacrylic resin, and derivatives thereof.

58. A cuff as claimed in claim 57, wherein the thermoplastic resin is one or more selected from a group composing of polyvinyl chloride, polyurethane resin, fluorocarbon resin, and silicone resin.

59. A cuff as claimed in claim 46, wherein the thickness of the flexible film is 0.1-500 mm.

60. A cuff as claimed in claim 59, wherein the thickness of the flexible film is 0.1-100 mm.

61. A cuff as claimed in claim 60, wherein the thickness of the flexible film is 0.1-50 mm.

62. A cuff as claimed in claim 61, wherein the thickness of the flexible film is 0.1-10 mm.

63. A cuff as claimed in claim 46, wherein the porous three-dimensional network structure of the second layer has an average pore diameter of 0.1-200 μm and an apparent density of from 0.01 to 1.0 g/cm³.

64. A cuff as claimed in claim 46, wherein the thickness of the porous three-dimensional network structure of the second layer is from 0.2 to 20 mm.

65. A cuff as claimed in claim 28, wherein one or more selected from a group composing of collagen Type I, collagen Type II, collagen Type III, collagen Type IV, atelocollagen, fibronectin, gelatin, hyaluronic acid, heparin, keratin acid, chondroitin, chondroitin sulfate, condroitin sulfate B, elastin, heparan sulfate, laminin, thrombospondin, hydronectin, osteonectin, entactin, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone is held in the porous three-dimensional network structure.

66. A cuff as claimed in claim 65, wherein one or more selected from a group composing of platelet-derived growth factor, epidermal growth factor, transforming growth factor-α, insulin-like growth factor, insulin-like growth factor binding proteins, hepatocyte growth factor, vascular endothelial proliferation growth factor, angiopoietin, nerve growth factor, brain-derived neurotrophic factor, ciliary neurotrophic factor, transforming growth factor-β, latent form transforming growth factor-β, activin, bone plasma proteins, fibrocyte growth factor, tumor growth factor-β, diploid fibrocyte growth factor, heparin-binding epidermal growth factor-like growth factor, schwannoma-derived growth factor, anfillegrin, betacellulin, epillegrin, lymphotoxin, erythropoietin, tumor necrosis factor-α, interleukin-1β, interleukin-6, interleukin-8, interleukin-17, interferon, antivirotic, antimicrobial agent, and antibacterial agent is further held in the porous three-dimensional network structure.

67. A cuff as claimed in claim 66, wherein cells are adhered on the porous three-dimensional network structure.

68. A cuff as claimed in claim 67, wherein said cells are cells of one or more kinds selected from a group composing of embryo-stem cell, vascular endothelial cell, mesodermal cell, smooth muscle cell, peripheral vessel cell, and mesothelial cell.

69. A cuff as claimed in claim 68, wherein the embryo-stem cell is dividing cell.

70. A biological implant covering member comprising a porous three-dimensional network structure which is made of a substrate resin composed of thermoplastic resin or thermosetting resin and has communication property, wherein the porous three-dimensional network structure has an average pore diameter of from 100 to 1000 μm and apparent density of from 0.01 to 0.5 g/cm³.

71. A biological implant covering member as claimed in claim 70, wherein the average pore diameter of said porous three-dimensional network structure is from 200 to 600 μm and the apparent density is from 0.01 to 0.5 g/cm³.

72. A biological implant covering member as claimed in claim 71, wherein the average pore diameter of said porous three-dimensional network structure is from 200 to 500 μm and the apparent density is from 0.01 to 0.5 g/cm³.

73. A biological implant covering member as claimed in any one of claims 70 through 72, wherein the apparent density of said porous three-dimensional network structure is from 0.05 to 0.3 g/cm³.

74. A biological implant covering member as claimed in claim 73, wherein the apparent density of said porous three-dimensional network structure is from 0.05 to 0.2 g/cm³.

75. A biological implant covering member as claimed in claim 70, wherein the contribution ratio of pores of 150-400 μm diameter in the average pore diameter of the porous three-dimensional network structure is 10% or more.

76. A biological implant covering member as claimed in claim 75, wherein the contribution ratio of pores of 150-400 μm diameter in the average pore diameter of the porous three-dimensional network structure is 20% or more.

77. A biological implant covering member as claimed in claim 76, wherein the contribution ratio of pores of 150-400 μm diameter in the average pore diameter of the porous three-dimensional network structure is 30% or more.

78. A biological implant covering member as claimed in claim 77, wherein the contribution ratio of pores of 150-400 μm diameter in the average pore diameter of the porous three-dimensional network structure is 40% or more.

79. A biological implant covering member as claimed in claim 78, wherein the contribution ratio of pores of 150-400 μm diameter in the average pore diameter of the porous three-dimensional network structure is 50% or more.

80. A biological implant covering member as claimed in claim 70, wherein the thickness of the porous three-dimensional network structure is from 0.5 to 500 mm.

81. A biological implant covering member as claimed in claim 80, wherein the thickness of the porous three-dimensional network structure is from 0.5 to 100 mm.

82. A biological implant covering member as claimed in claim 81, wherein the thickness of the porous three-dimensional network structure is from 0.5 to 50 mm.

83. A biological implant covering member as claimed in claim 82, wherein the thickness of the porous three-dimensional network structure is from 0.5 to 10 mm.

84. A biological implant covering member as claimed in claim 83, wherein the thickness of the porous three-dimensional network structure is from 0.5 to 5 mm.

85. A biological implant covering member as claimed in claim 70, wherein said substrate resin is one or more selected from a group composing of polyurethane resin, polyamide resin, polylactide resin, polymalate resin, polyglycolate resin, polyolefin resin, polyester resin, fluorocarbon resin, urea resin, phenol resin, epoxy resin, polyimide resin, acrylic resin, methacrylic resin, and derivatives thereof.

86. A biological implant covering member as claimed in claim 85, wherein the substrate resin is polyurethane resin.

87. A biological implant covering member as claimed in claim 86, wherein the polyurethane resin is segmented polyurethane resin.

88. A biological implant covering member as claimed in claim 70, wherein the biological implant covering member is a lamination of a first layer composed of the porous three-dimensional network structure and a second layer different from the first layer.

89. A biological implant covering member as claimed in claim 70, wherein one or more selected from a group composing of collagen Type I, collagen Type II, collagen Type III, collagen Type IV, atelocollagen, fibronectin, gelatin, hyaluronic acid, heparin, keratin acid, chondroitin, chondroitin sulfate, condroitin sulfate B, elastin, heparan sulfate, laminin, thrombospondin, hydronectin, osteonectin, entactin, copolymer of hydroxyethyl methacrylate and dimethylaminoethyl methacrylate, copolymer of hydroxyethyl methacrylate and methacrylic acid, alginic acid, polyacrylamide, polydimethylacrylamide, and polyvinyl pyrrolidone is held in the porous three-dimensional network structure.

90. A biological implant covering member as claimed in claim 89, wherein one or more selected from a group composing of platelet-derived growth factor, epidermal growth factor, transforming growth factor-α, insulin-like growth factor, insulin-like growth factor binding proteins, hepatocyte growth factor, vascular endothelial proliferation growth factor, angiopoietin, nerve growth factor, brain-derived neurotrophic factor, ciliary neurotrophic factor, transforming growth factor-β, latent form transforming growth factor-β, activin, bone plasma proteins, fibrocyte growth factor, tumor growth factor-β, diploid fibrocyte growth factor, heparin-binding epidermal growth factor-like growth factor, schwannoma-derived growth factor, anfillegrin, betacellulin, epighrelin, lymphotoxin, erythropoietin, tumor necrosis factor-α, interleukin-1β, interleukin-6, interleukin-8, interleukin-17, interferon, antivirotic, antimicrobial agent, and antibacterial agent is further held in the porous three-dimensional network structure.

91. A biological implant covering member as claimed in claim 90, wherein cells are adhered on the porous three-dimensional network structure.

92. A biological implant covering member as claimed in claim 91, wherein said cells are cells of one or more kinds selected from a group composing of embryo-stem cell, vascular endothelial cell, mesodermal cell, smooth muscle cell, peripheral vessel cell, and mesothelial cell.

93. A biological implant covering member as claimed in claim 92, wherein the embryo-stem cell is dividing cell.
